(19) United States
(12) Patent Application Publication
(10) Pub. No.: US 2025/0253015 A1
(43) Pub. Date: Aug. 7, 2025

(54) REAL-TIME PREDICTION OF CONFIDENCE AND ERROR PARAMETERS ASSOCIATED WITH TEST RESULTS, ESTIMATES, AND FORECASTS

(71) Applicant: Anaclara Systems Limited, Little Kimble, Buckinghamshire (GB)

(72) Inventors: Andrew DEONARINE, New York City, NY (US); Railton FRITH, Little Kimble (GB); Wanlu WANG, Singapore (SG); Jason LONG, Cambridge (GB)

(73) Assignee: Anaclara Systems Limited, Little Kimble, Buckinghamshire (GB)

(21) Appl. No.: 18/856,177

(22) PCT Filed: Apr. 11, 2023
(86) PCT No.: PCT/US23/18112

§ 371 Date: Oct. 11, 2024

Related U.S. Application Data
(60) Provisional application No. 63/329,829, filed on Apr. 11, 2022.

(51) Int. Cl. G16H 10/20; G06F 17/18

(52) U.S. Cl. CPC G16H 10/20; G06F 17/18

(57) ABSTRACT

A method for determining the error associated with results, such as medical test output, machine-learning or artificial intelligence predictions and classifications, financial predictions, and engineering models, is disclosed. These results are calculated using population-level data and individual-level data. The results can be communicated and used to generate actionable reports and data for use in connection with decision support tools. These data can be used to determine if a test result is likely to be correct or incorrect, and whether the test should be accepted, repeated, or supplemented with another test/analysis. This determination can be made using decision rules and analyses which can be organized to provide explainability of the status of the results were determined to be true, false, or other state.

### FIELD OF THE INVENTION

The invention relates to a method for determining the error associated with test results, estimates and forecasts, such as medical test output, machine-learning or artificial intelligence predictions and classifications, financial predictions, and engineering models. Such results are calculated using population-level data and individual-level data by a method comprising the following steps: obtaining population-level data of characteristics, disease incidence, prevalence, and test results at various geographical levels (such as census tracts, neighborhoods cities, states, and nationally) and various demographic groups; calculating positive predictive values, negative predictive values, false discovery rates, and other epidemiological measures of test performance; obtaining individual-level data pertaining to results over time; applying a model (which can include machine-learning models) to predict the next test result given previous test results and the results of other test results in that individual; combining the population-level measures and individual level measures (optionally using machine-learning methods) to produce an estimate of the probability that a test result is correct or incorrect (such as an odds ratio or risk ratio); calculating other parameters associated with the error estimation such as validity, robustness, sensitivity, performance, and explainability. The method further includes a step of communicating the results to other computer systems and using those results to generate actionable reports and data that can be used in decision support tools. These data can be used to determine if a test result is likely to be correct or incorrect, and whether the test should be accepted, repeated, or supplemented with another test/analysis.

### BACKGROUND OF THE INVENTION

It is estimated that there are over 250,000 deaths per year in the United States due to medical errors, and medical errors are reported to be the third leading cause of death in America [https://pubmed.ncbi.nlm.nih.gov/28186008/]. There are several types of medical errors, including inaccurate test results, medical prescription errors, and drug interactions. Developing methods to avoid medical errors that can be used in computerized medical information systems is a major area of research. Medical data such as laboratory results are used to diagnose diseases, guide prescriptions, and guide medical decisions.

Several statistical measures are used to assess the accuracy of clinical instruments such as medical tests, predictive algorithms, or decision support tools. These measures include values such as positive predictive value (PPV), negative predictive value (NPV), and false discovery rate (FDR), and other measures such as the receiver-operator area under the curve (ROAUC). While these values are generally calculated using epidemiological data at a national or international level, it is possible they also vary with higher geographical resolution, such as states, cities, counties, and census tracts/blocks.

A method of calculating these epidemiological measures at the highest geographical resolution could help the healthcare system and similar industries better interpret the results of a clinical instrument using local epidemiological measures. This is particularly important during a pandemic, where PPV, NPV, and FDR can vary significantly based on the rate of the disease in the public, and that these rates will vary continuously due to vaccination rates, new emergent strains, and other factors.

### BRIEF SUMMARY OF INVENTION

The invention relates to a method of calculating various parameters associated with tests results, estimates and forecasts using statistical and related machine-learning methods for various demographic subgroups. In one embodiment, the invention relates to a method of calculating various parameters associated with epidemiological parameters normally associated with medical tests, such as PPV, NPV, ROAUC, and prevalence, using statistical and related machine-learning methods for various demographic subgroups. The method can be used for determining if the results of a laboratory test, such as a COVID-19 antibody test, is likely to be valid where the rates of diseases vary for different socioeconomic groups, and therefore the prevalence of diseases change. The PPV, NPV, FDR, and other parameters are used to determine if a laboratory test, the results of a computer analysis, or machine learning output are likely to be valid or not, and whether a laboratory test needs to be repeated, supplemented, or accepted.

### BRIEF DESCRIPTION OF THE DRAWINGS

The various features of the present invention and the manner of attaining them will be described in greater detail with reference to the following description, claims, drawings, wherein like designations denote like elements.

FIG. 1 depicts a flowscheme of data acquisition, extraction, transformation, and loading processes, together with data analysis and presentation, according to one embodiment of the invention.

FIG. 2 depicts a flowscheme of a data extraction, transformation, data validation and loading process according to one embodiment of the invention.

FIG. 3 depicts a flowscheme of an ensemble machine learning module according to one embodiment of the invention.

FIG. 4 depicts a flowscheme of model data and input mapping processes according to one embodiment of the invention.

FIG. 5 depicts a flowscheme of calculation of error parameters (EPs) and error statistical parameters (ESPs) using input data according to one embodiment of the invention.

FIG. 6 depicts a flowscheme of a process for determining the validity of a result using EPs and ESPs according to one embodiment of the invention.

FIG. 7 depicts a flowscheme for storing and encoding EPs, ESPs, and output into a final report according to one embodiment of the invention.

FIG. 8 depicts a flowscheme for generating a report based on EPs, ESPs, and results stored in electronic format according to one embodiment of the invention.

FIG. 9 depicts application of the method to a system for facial recognition according to one embodiment of the invention.

FIG. 10 depicts application of the method to a system to COVID-19 antibody testing according to one embodiment of the invention.

FIG. 11 depicts a schematic of a computer system suitable for performing the method for calculating EPs and ESPs according to one embodiment of the invention.

FIG. 12 is a graph illustrating the use of positive predictive value (PPV) to determine if a COVID-19 PCR test should be repeated or not using a PPV threshold.

FIGS. 13a-13f are graphs of state-level positive predictive values (PPVs) forecasted using Bayesian methods, autoregressive integrated moving average (ARIMA) or an artificial neural network (ANN). A PPV threshold of 60% is illustrated by the dash horizontal line (if a PPV of the test on the given day is below the threshold, then repeat the test, and if it's above, no repeat is required).

FIGS. 14a-14f are graphs of county-level positive predictive values (PPVs) forecasted using Bayesian methods, autoregressive integrated moving average (ARIMA) or an artificial neural network (ANN). A PPV threshold of 60% is illustrated (if a PPV of the test on the given day is below the threshold, then repeat the test, and if it's above, no repeat is required).

### DETAILED DESCRIPTION OF THE INVENTION

The invention relates to a method of calculating various parameters associated with tests results, estimates and forecasts using statistical and related machine-learning methods for various demographic subgroups.

In one embodiment relating to medical testing, the invention relates to a method of calculating various parameters associated with epidemiological parameters normally associated with medical tests, such as PPV, NPV, and prevalence, using statistical and related machine-learning methods for various demographic subgroups.

The machine-learning (ML) computer program module can use ML or artificial intelligence (AI) algorithms, and it will be provided with a plurality of various data sets of test results which have been confirmed as likely to be valid for a particular result, which will be considered the baseline data points. These baseline data points provide the programmed computer of the “valid” conditions for a particular result, for example, test results for COVID-19 that are likely to be “valid.” The data sets will further comprise examples that are indicated as a “negative” or results not likely to be a “valid” for a particular result. Based on the data sets, the programmed computer will “learn” to discern between results likely to be valid and results not likely to be valid. This process of “learning” will be repeated for each individual result and population. The programmed computer may be reprogrammed to reflect changes in a data set or population. As the programmed computer “learns” its results will eventually only be randomly viewed by humans to confirm that it is operating within programmed parameters as well as to minimize false positive results.

The method can be used for determining if the results of a laboratory test, such as a COVID-19 antibody test, is likely to be valid where the rates of diseases vary for different socioeconomic groups, and therefore the prevalence of diseases change. The PPV, NPV, FDR, and other parameters are used to determine if a laboratory test, the results of a computer analysis, or machine learning output are likely to be valid or not, and whether a laboratory test needs to be repeated, supplemented, or accepted.

In other embodiments, the method can be applied to a system for facial recognition.

In other embodiments of the invention, the method can be applied to any type or test result, estimates and forecasts such as machine-learning or artificial intelligence predictions and classifications, financial predictions, and engineering models, to determine if a result is likely to be correct or incorrect, and whether the test should be accepted, repeated, or supplemented with another test/analysis.

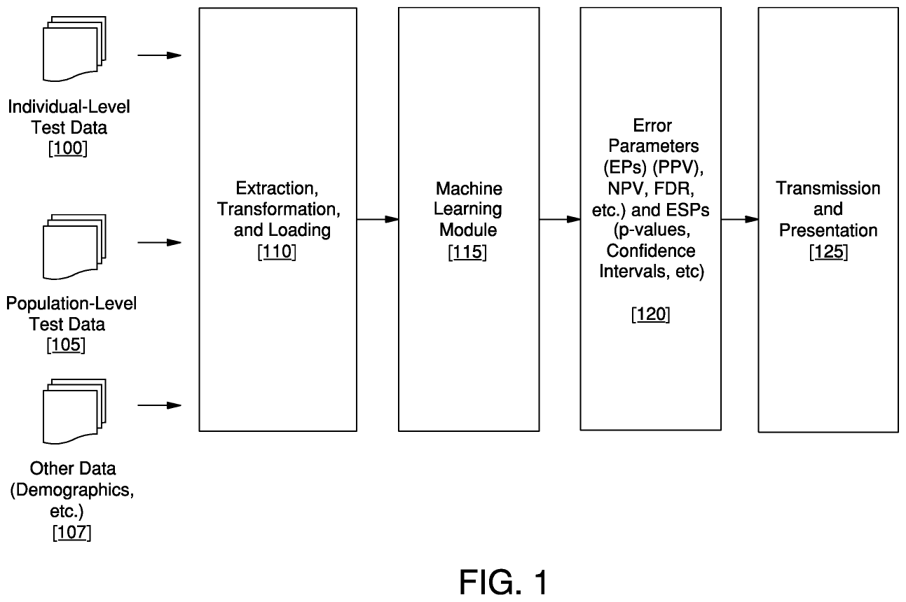

Turning to the figures, FIG. 1 depicts a flowscheme of data acquisition, extraction, transformation, and loading processes, together with data analysis and presentation, according to one embodiment of the invention. The method may use individual-level data [100] and/or population-level data [105] and may use other associated data (such as demographics, test populations, etc.) [107]. According to one embodiment of the invention, these data can be processed using an extraction, transformation, and loading pipeline [110]. Once processed, these data can subsequently be mapped to various model inputs and processed using a machine-learning module [115].

According to one embodiment of the invention, the method calculates prevalence, sensitivity, specificity, positive predictive value (PPV), negative predictive value (NPV), false-discovery rate (FDR), and false-omission rate (FOR), likelihood ratio (LR), odds ratio (OR), receiver-operator area under the curve (ROAUC), and bias, herein referred to as error parameters (EPs) and p-values, confidence intervals, multiple-comparison adjusted p-values, sample size, and entropy, herein referred to as error-related statistical parameters (ESPs) after transforming the raw output [120]. Finally, this information is then encoded and then transmitted and presented to end-users [125].

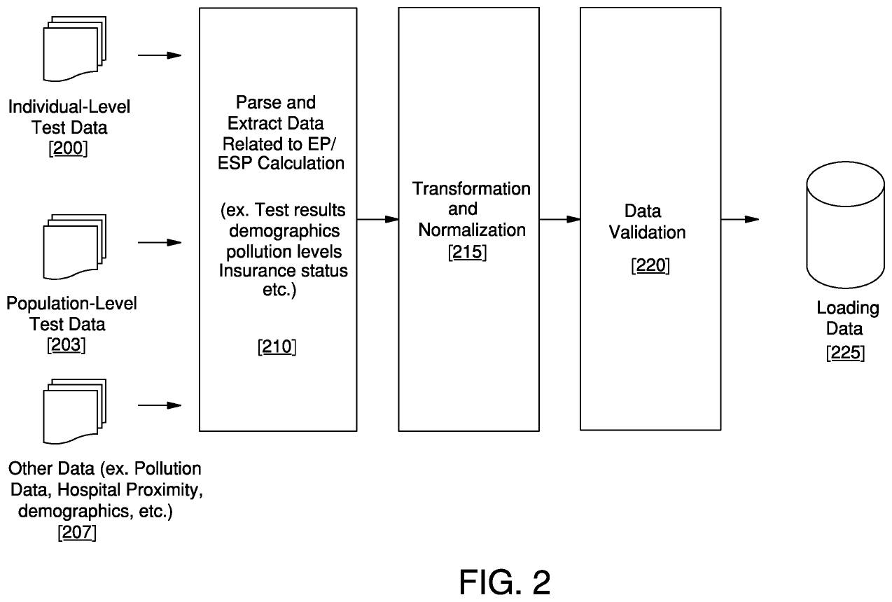

FIG. 2 depicts a flowscheme of a data extraction, transformation, data validation and loading process according to one embodiment of the invention. The method may use individual level data [200] which can include single test results, or laboratory test values over time, as well as associated laboratory values, demographics and other information. The method can also use population level test data [203] which can comprise population-level laboratory results (such as median and mean laboratory values) over time. Other data may also be used such as for example and without limitation pollution data, hospital proximity, economic data, medical test results and demographics [207]. Data [200], [203], [207] are extracted at [210]. These sources of data can consist of databases, scanned paper results, computer files (such as but not limited to spreadsheets, binary data, text files, JSON, or XML). Next, data are transformed and normalized into a format that can be used by the machine learning module (AI). Transformation involves turning extracted data, such as electronic text, into numerical values corresponding to laboratory or test values [215]. Normalization is then performed by taking numerical values and ensuring that they are on the correct scale. For instance, a laboratory test corresponding to white blood cells could be scaled to international standard units like cells per liter. Then, the data are validated [220], which involves assessing whether the value falls within the known and allowable range of values for the given source (for example, a laboratory test) and for the appropriate scale corresponding to the value. Finally, data is then loaded [225] into a computer system for analysis by the machine learning (ML) artificial intelligence (AI) module.

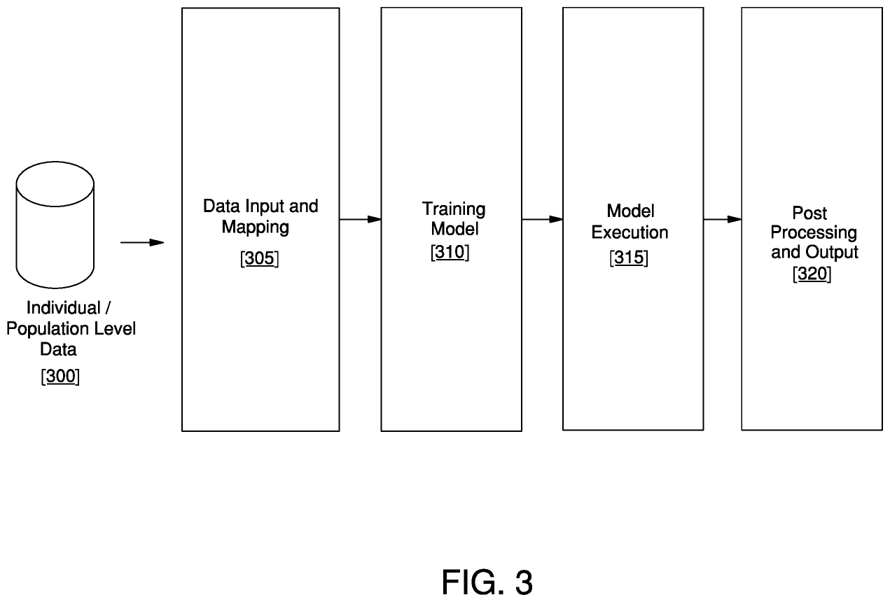

FIG. 3 depicts a flowscheme of an ensemble machine learning module according to one embodiment of the invention. At [300], individual and population-level data that have undergone extraction, transformation, and loading are mapped to machine-learning inputs through the process of input mapping [305]. Using this mapped data, the machine-learning module is then trained on a training set of data [310], and the model is then tested using a testing set. Finally, the model is executed [315] using the whole dataset, producing raw output data that is then post-processed to produce information such as EPs and ESPs [320].

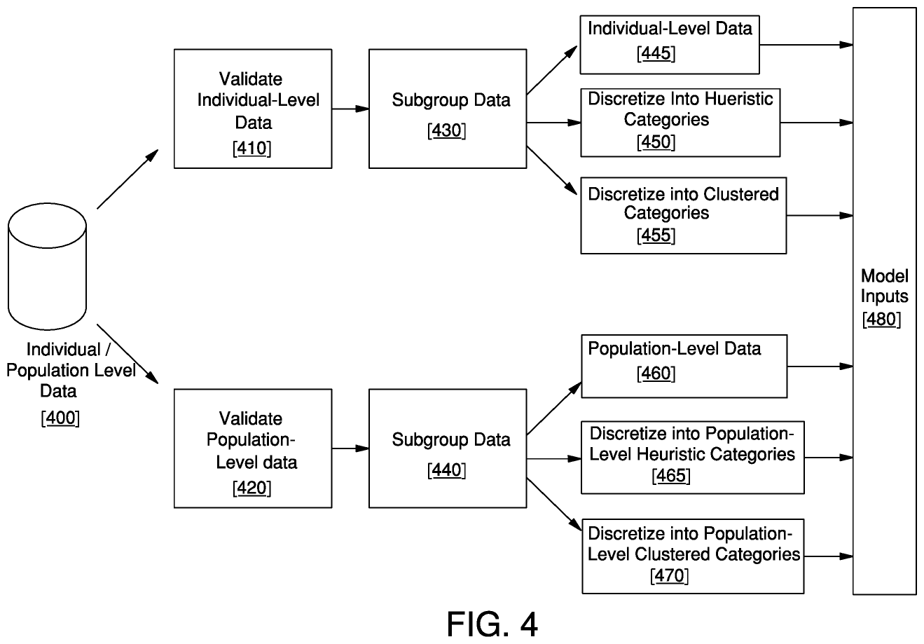

FIG. 4 depicts a flowscheme of model data and input mapping processes according to one embodiment of the invention. At [400], individual and population-level data are split into individual-level [410] and population-level data [420] based on meta-data and annotations associated with each measure. The data are then subdivided into different subgroups by various categories, such as ethnicity, age, sex, economic group, and location. This subgrouping is done for individual [430] and population-level [440] data. Once this subgrouping has been performed, the data can then be kept as un-transformed individual-level data [445], or discretized into statistical categories such as tertiles, quartiles, or quintiles [450], or into machine-learning derived clusters such as hierarchical clustering-derived groups [455]. Similarly for population-level data, direct values can be used as model inputs to the machine-learning model [460], discretized into population-level heuristic categories like tertiles, quartiles, or quintiles [465], or into machine-learning derived clustered categories [470]. Finally, all of the different information from [445, 450, 455, 460, 465, and 470] constitute the set of model inputs [480] according to this embodiment of the invention.

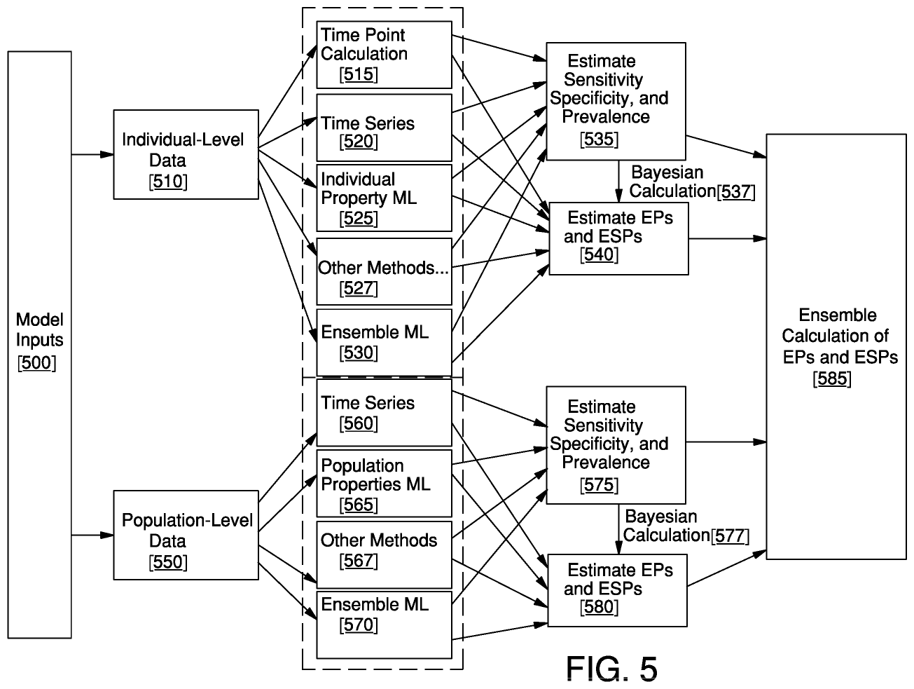

FIG. 5 depicts a flowscheme of calculation of error parameters (EPs) and error statistical parameters (ESPs) using input data according to one embodiment of the invention. Model input data [500] are divided into individual-level data [510] and population-level [550] data. There are several methods for calculating EPs and ESPs that are known to those skilled in the art, and later methods may be developed which are suitable for use in the method of the invention. Exemplary methods for calculating EPs and ESPs include but are not limited to statistical regression models, machine-learning based approaches, time series analyses, or ensemble models which use a combination of regression, machine-learning, and other approaches, as well as other approaches not listed here.

Different approaches can be used to calculate EPs and ESPs at the individual-level calculation module [532]. EPs and ESPs can be calculated for individual-level results at a current time point t using the true positive, true negative, false positive, and false negative rates calculated from individual-level data [510]. Another method uses time series data at the individual level, in which a regression analysis is performed to calculate EPs and ESPs at the current time point using EPs and ESPs at previous time points as inputs into a statistical model, such as a linear regression, multiple linear regression, logistic regression (with the output being a discrete class associated with an EP and/or ESP), ARIMA model, or another time-series modelling approach [520]. Alternatively, the properties of the individual-level data, such as the proportion of males, females, percentage in low socioeconomic groups, or other features, can be used as input to a machine learning model [525] such as a logistic regression calculated using a maximum-likelihood estimation method, an elastic net regression, xgboost, or another approach, which produces EPs and associated ESPs as output. Alternatively, other methods such as alternative machine-learning, neural networks, deep learning, or statistical models could be used to calculate EPs and ESPs [527]. An ensemble machine learning model [530] can also be used to calculate EPs and ESPs. Together these example calculation methods are part of the individual-level calculation module [532].

In this ensemble model, Elastic Net could be used to calculate current EPs and/or ESPs using previous EPs and/or ESPs at various time points, and neural networks with different architectures. The results from these different models could then be combined using bagging (bootstrap aggregating), Bayesian model combination, or Bayesian model averaging. These four calculation methods [515, 520, 525, 527, 530] can be used to estimate sensitivity, specificity, and prevalence values [535], as well as estimate all EPs and ESPs directly [540]. Additionally, EPs and ESPs such as PPV and NPV can be calculated from sensitivity, specificity, and prevalence [535] using a Bayesian calculation method [537] illustrated in the following equation for positive predictive value:

$$Bayesian calculation of PPV .$$

$$P P V = \frac{Sensitivity \times Prevalence}{\begin{matrix}Sensitivity \times Prevalence + \\ ( 1 - Specificity ) \times ( 1 - Prevalence )\end{matrix}} . \qquad Equation A$$

Using this approach, 10 different estimates for EPs and ESPs [540](PPV, NPV, FDR, etc) can be produced from individual-level data [510].

Similarly, population-level data and measurements can be used to create estimates of EPs and ESPs. Time series measurements of EPs and ESPs at the population-level data [550] can be used to predict the current EPs and/or ESPs using simple linear regression analysis, general linear models, ARIMA, or similar approaches [560]. Population-level properties, such as sex percentages, income, ethnicity, and statistics can be used as inputs to machine-learning models to estimate EPs and/or ESPs with methods such as logistic regression calculated using maximum likelihood estimation, elastic net, xgboost, or other approaches [565]. Alternatively, different methods (such as other machine learning, neural network, or deep-learning approaches) can be used to calculate EPs and/or ESPs [567]. Similar to the ensemble model used with individual-level data, ensemble models (such as elastic net and neural networks) can be used to produce estimates of EPs and/or ESPs with bagging [570]. These population-level data calculation methods are included in the Population-Level Calculation Module [572]. These methods [560, 565, 567, 570] can then be used to estimate the EPs and/or ESPs sensitivity, specificity, and prevalence, which can then be used to estimate other EPs and/or ESPs like PPV, NPV, FDR, OR, and ROAUC [580] using Bayesian methods [577] as with the individual-level calculation ([537]). Alternatively, Estimate EPs and/or ESPs [580] can be estimated directly using these different methods [560, 565, 567, 570], and through Bayesian methods resulting in estimates of EPs and/or ESPs. Finally, EP and/or ESP estimates [540] and [580] can be used to produce an Ensemble Calculation of EPs and ESPs [585] by taking a simple average of the EPs and ESPs, a weighted average, a variance-weighted mean, or using another approach, while also showing the range of values. Outliers, such as the most extreme EP and/or ESP values can be excluded from the mean calculation. Median values can also be returned for each EP and/or ESP as an estimate of the true EP and/or ESP.

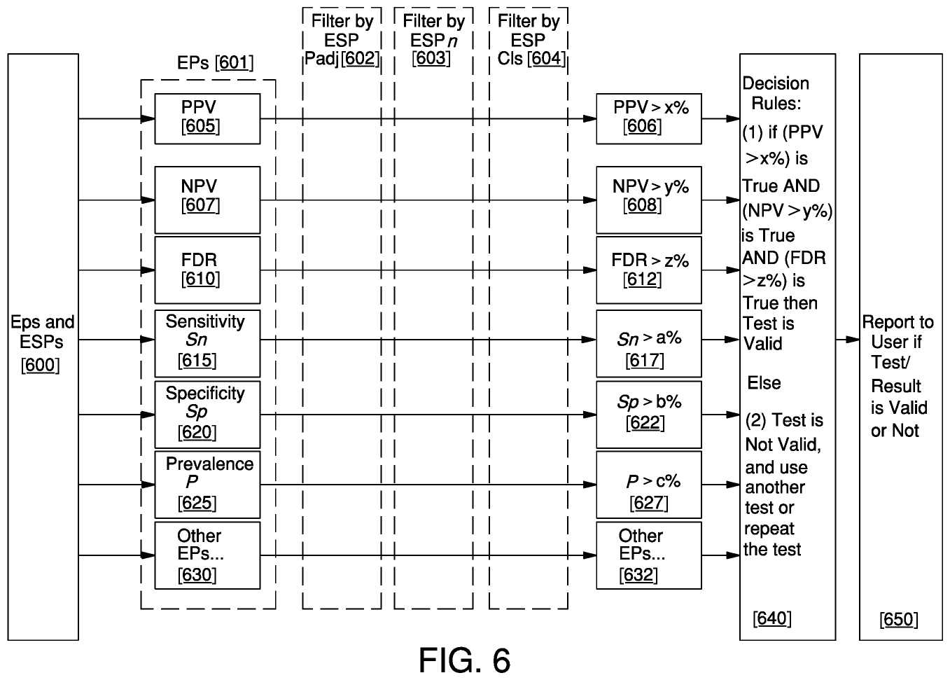

FIG. 6 depicts a flowscheme of a process for determining the validity of a result using EPs and ESPs according to one embodiment of the invention. EPs [601] and ESPs [602, 603, 604] input from the Ensemble Calculation [585] are divided into separate error parameter measures, with PPV [605], NPV [607], FDR [610], sensitivity [615], specificity [620], prevalence [625] illustrated, and other error parameters possible [630]. Each error parameter EP is filtered by p-value (padj) [602], by sample size n [603], and the range of the confidence intervals for each estimate (ex. assessing if a confidence interval includes 1 for an odds ratio) [604]. Once the EPs are filtered through [602, 603, 604], then a set of decision rules are applied to each EP individually. Satisfying the decision rule will result in a value of “true”, and not satisfying the rule will result in a value of “false”. These decision rules can be applied to individual EPs [606, 608, 612, 617, 622, 627, 632] using various cutoff values such as x % [606], y % [608], z % [612], a % [617], b % [622], c % [632]. These values can be varied by the user, or set to common defaults such as 90%, 90%, 5%, 90%, 90%, and 0.05 respectively. Once these conditions in [606, 608, 612, 617, 622, 627, 632] have been evaluated to true or false, decision rules can be applied to determine if a test result or model output is valid or not [640]. Sample decision rules include if the PPV rule is true [606], the NPV rule is true [608], and the FDR condition is true [612], then report the test result as valid [650].

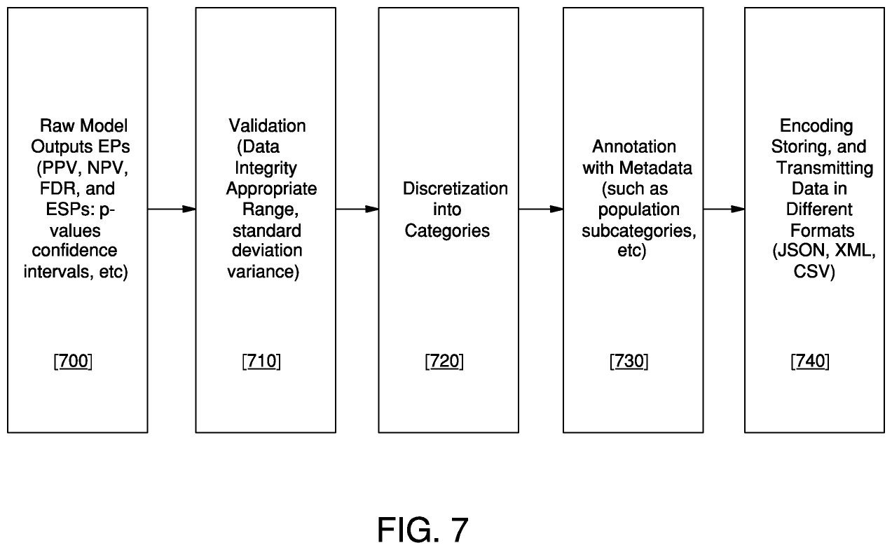

FIG. 7 depicts a flowscheme for storing and encoding EPs, ESPs, and output into a final report according to one embodiment of the invention. Once the EPs and ESPs [600] and a report [650] have been generated [700], the data are then validated to ensure that the values fall within appropriate ranges, have appropriate confidence intervals, and are valid numbers [710]. The values can then be discretized into further categories such as tertiles corresponding to low, medium, and high (or quintiles such as low, medium low, medium, medium high, or high) [720]. These categories can then be annotated with additional information (including designations such as “low”, “medium”, or “high”) [730]. This information is then stored in different computational formats such as CSVs, XML, or JSON [740].

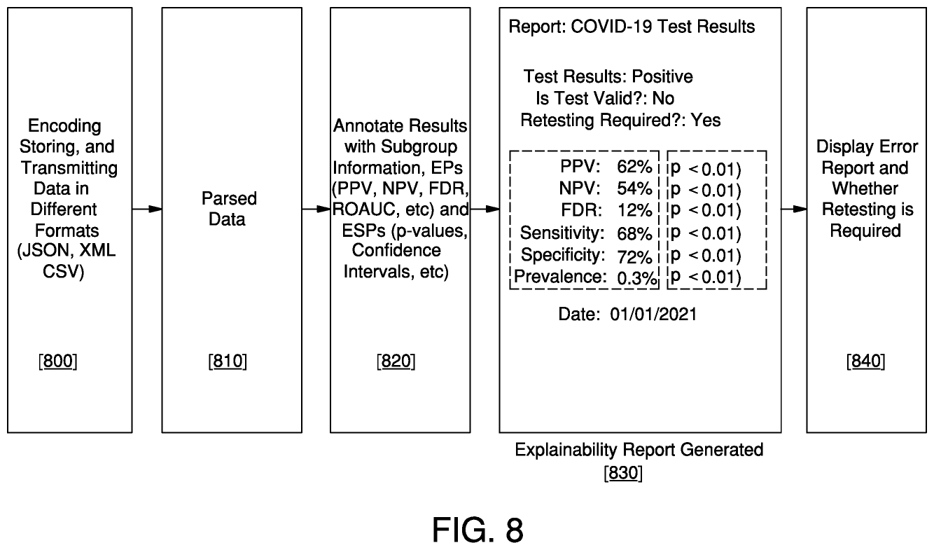

FIG. 8 depicts a flowscheme for generating a report based on EPs, ESPs, and results stored in electronic format according to one embodiment of the invention. Once the data have been encoded and stored in non-volatile or volatile memory [800], they can be transmitted to another device over a network or processed for presentation on the same or another computing device. The data are parsed [810] using an appropriate parser for the data format (such as a JSON parser for JSON or XML parser for XML). The extracted values will also consist of associated statistical parameters (such as confidence intervals and p-values), and metadata [820]. The explainability of the outcome is then provided using an appropriate visualization, which can include a numerical display, bar chart, line chart, or other method can then be selected to display the results and decision rule outcomes [830]. Finally, the explainability of the data, which can include an appropriate visualization can be integrated into an electronic report, which can be a readout in a separate software application, in a custom-made software program. This will include an annotation or explanation of whether or not a test should be repeated, another test performed to supplement the current test, or whether the test result is valid [840].

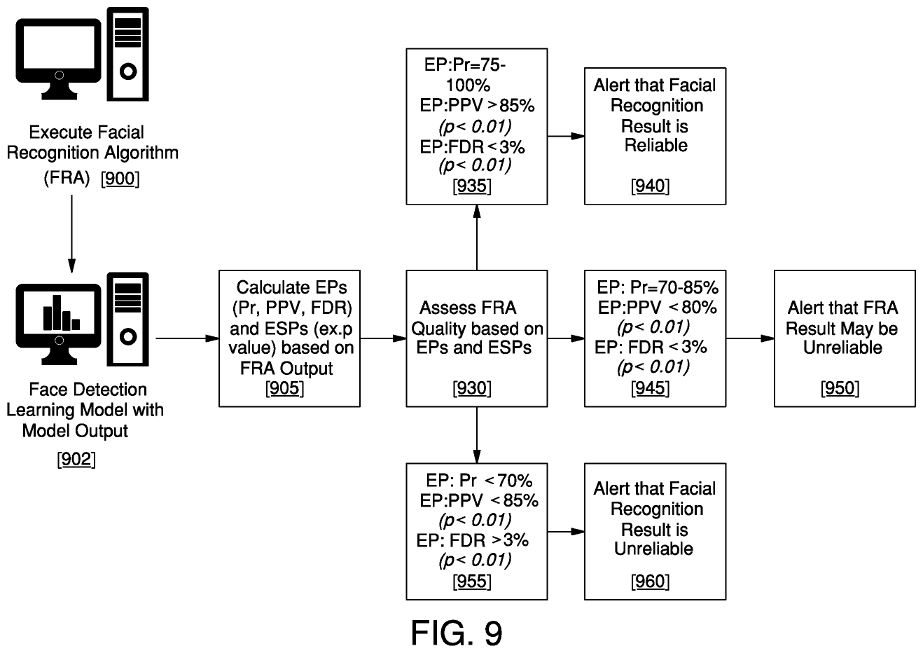

FIG. 9 depicts application of the method to a system for facial recognition according to one embodiment of the invention. In some facial recognition systems, faces are detected at lower rates for certain ethnicities and races. Using a corpus of photographs of people from different ethnic and racial backgrounds, a facial recognition algorithm (FRA) is executed [900], and the results and outputs [902] are then used to calculate EPs and ESPs based on this output [905]. Once the EPs and ESPs are calculated, they can then be used to determine if the facial recognition result is reliable [940], may be unreliable [950], or is unreliable [960]. To do this, decision rules are applied to the EPs and ESPs. In the case of the facial recognition result being reliable, the corresponding EP (and optionally ESP) conditions need to be true [935]. For the result being classified as possibly unreliable [950], some of the conditions outlined in [945] should be false. Finally, for the facial recognition result to be flagged as unreliable, the EP (and optionally ESP) conditions in [955] should all be evaluated to false [960].

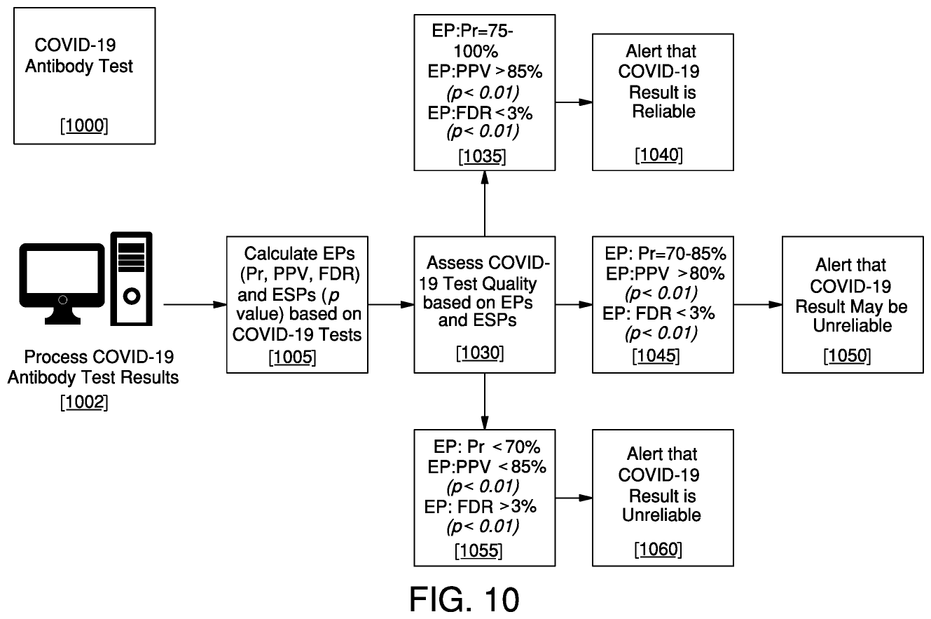

FIG. 10 depicts application of the method to a system to COVID-19 antibody testing according to one embodiment of the invention. In this embodiment, a COVID-19 antibody test is performed [1000], and the results are processed to determine if the result returned is “true” or “false” [1002]. Using the results from the COVID-19 antibody test [1000] in conjunction with other test results such as a COVID-19 PCR results, white blood cell count, or other data, EPs and ESPs are calculated [1005]. The COVID-19 test results are then assessed in conjunction with the EPs (and optionally ESPs) [1030] to result in three possible outcomes: the COVID-19 antibody test result looks reliable [1040], the result may be unreliable [1050], or the result is unreliable [1060]. For the test result to be flagged as likely reliable [1040], the conditions for the EPs (and optionally ESPs) need to all be evaluated to “true” [1035]. To indicate that the results may be unreliable, some of the decision rules/conditions which use EPs (and optionally ESPs) in [1045] need to be false. Finally, to indicate that the results are likely to be unreliable [1060], the corresponding decision rules in [1055] for should all be found to be false.

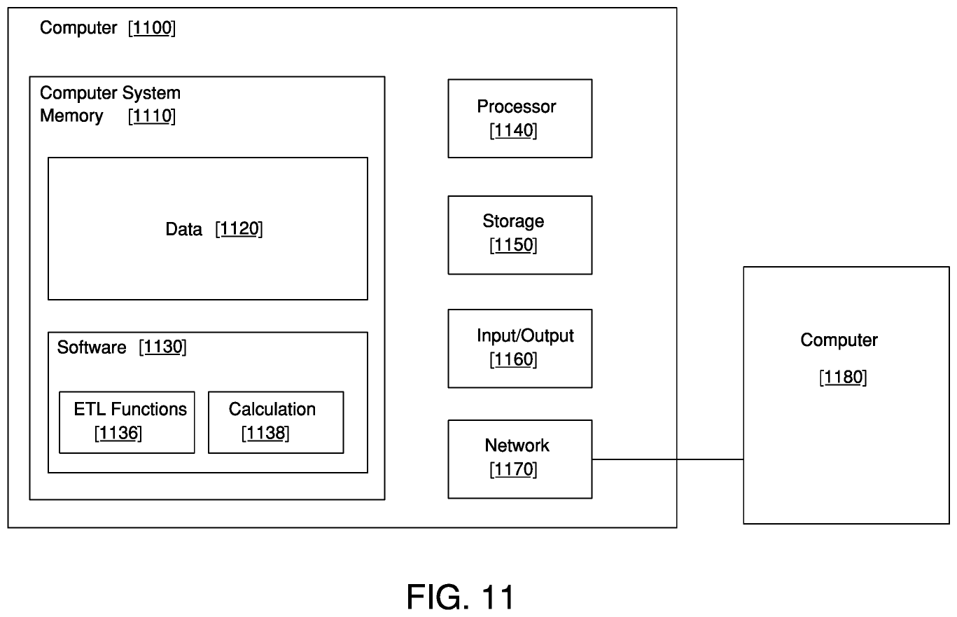

FIG. 11 depicts a schematic of a computer system suitable for performing the method for calculating EPs and ESPs according to one embodiment of the invention. A computer system [1100] comprises computer memory [1110] for storing data [1120] to calculate EPs and ESPs, and computer code to perform calculations [1130], which includes extraction, transformation, and loading (ETL) methods to process data [1136], and a calculation module [1138] which calculates the EPs and ESPs and then stores them in memory [1110]. All computational operations in the computer code are executed on one or more computer processor(s), co-processors, or AI/ML optimized processors, or calculation devices [1140], and data can be saved and stored using computer storage [1150]. These calculations can be performed in series or in parallel depending on the configuration of the processors and/or calculation devices. An input/output module is used to load data into memory and retrieve it from memory to transmit to another computer [1180] using a network connection [1170]. In one embodiment, the computer system comprises a single computer memory, processor and storage. In other embodiments, the computer memory, processor and storage may be found in a plurality of computer systems, where the computer systems are in communication with each other.

### EXAMPLE

In the United States, COVID-19 pandemic cases have been logged in a central database by Johns Hopkins University since Jan. 22, 2020 and are tabulated at the county level. Counties are a smaller geographical division than state and can cover a more granular picture of how the pandemic is evolving across America. We can calculate the (crude) prevalence of COVID-19 by dividing the number of cases in a county by the population of that county.

Positive predictive value (PPV) is an indicator of test quality and is the probability that someone who has a positive test actually has a disease. PPV can be calculated using Bayesian methods from Equation A as seen in Para. [0039].

PPV over the course of the pandemic for the COVID-19 PCR test was calculated using the method of the invention and forecast forward 50 days. The PPV depends on the prevalence of a test in a particular region and can therefore vary over time and location during the course of an outbreak, epidemic, or pandemic. A decision system was devised in which a PPV below 60% for a given region (where a region can be a county or state) requires a test to be repeated. When the PPV rises above 60%, it does not need to be repeated. Forecasting methods, such as statistical regression, can be used to determine if the PPV in a particular region will rise above the 60% PPV threshold or stay below it for the immediate future. This can be important to know since it will allow local governments to budget for additional tests, and then repeating tests will no longer be required.

The COVID-19 PCR test has a sensitivity of 70% and specificity of 95% (values taken from: https://www.bmj.com/content/bmj/369/bmj.m1808.full.pdf). Using the COVID-19 case numbers for each county and state obtained from Johns Hopkins University (https://github.com/CSSEGISandData/COVID-19), a crude prevalence value was calculated by dividing the number of COVID-19 cases in a particular location by the total population in that county or state. The total population numbers were obtained from the US Census (https://www2.census.gov/programs-surveys/popest/datasets/2010-2020/national/totals/nst-est2020-alldata.csv). Population counts were matched to the corresponding number of cases for each county or state using the FIPS (Federal Information Processing Standard) code. Prevalence was calculated for each day of the pandemic in America (since Jan. 22, 2020, according to the Johns Hopkins Database). For each day PPV was calculated using Equation “A” creating a time series of PPV values for each day.

Once the PPV values were obtained for each day of the COVID-19 pandemic, the PPV was then “forecasted” for 50 days into the future from Oct. 25, 2021. To calculate these forecasted values for future dates, two approaches were used: (1) autoregressive integrated moving average (ARIMA), and (2) a neural network (ANN). To complete the ARIMA forecasts, a computer program was written in Python 3.9 to take the previous PPV values since Jan. 22, 2020 and forecast for 50 future days from Oct. 25, 2021. The “statsmodels” library was used to calculate the ARIMA model fit and resulting predictions. For the ANN, the “keras” library was used in Python 3.9 and a sequential model with three, dense interconnected neural network layers was used with 100, 8, and 1 node in each layer respectively. Once the ANN was trained, forecasts were performed for the future 50 days as with the ARIMA regression model.

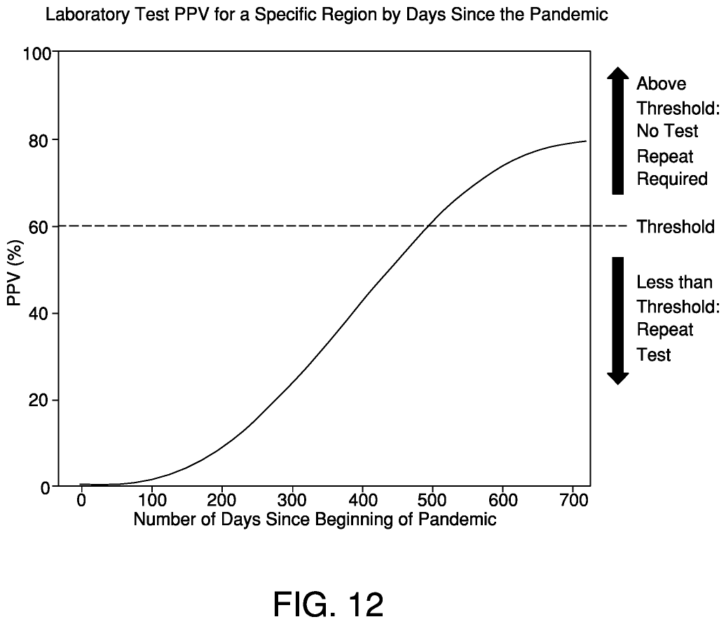

Once the forecasts were completed, a clinical heuristic was created to determine if a medical test needed to be repeated based on PPV value. PPV can reflect the reliability of a test result and is normally taken together with many other test parameters, such as negative predictive value (NPV), false discovery rate, and other features. In this case a heuristic was created to illustrate the approach: a PPV<60% requires a test to be repeated, while a PPV>=60% indicates the test does not need to be repeated. This scheme is illustrated in FIG. 12.

### RESULTS

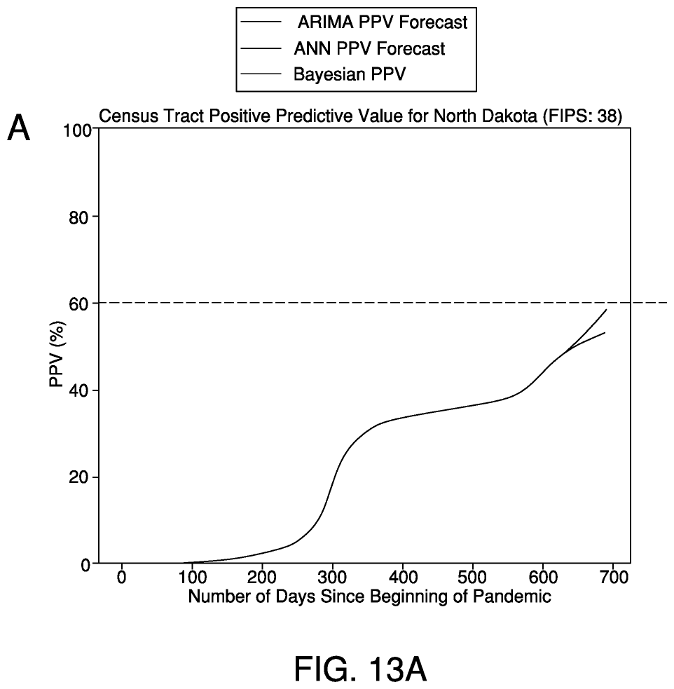

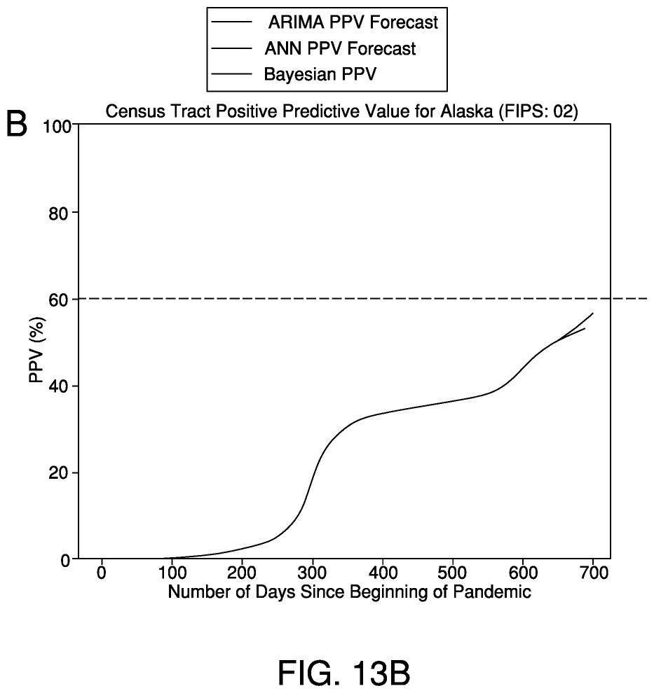

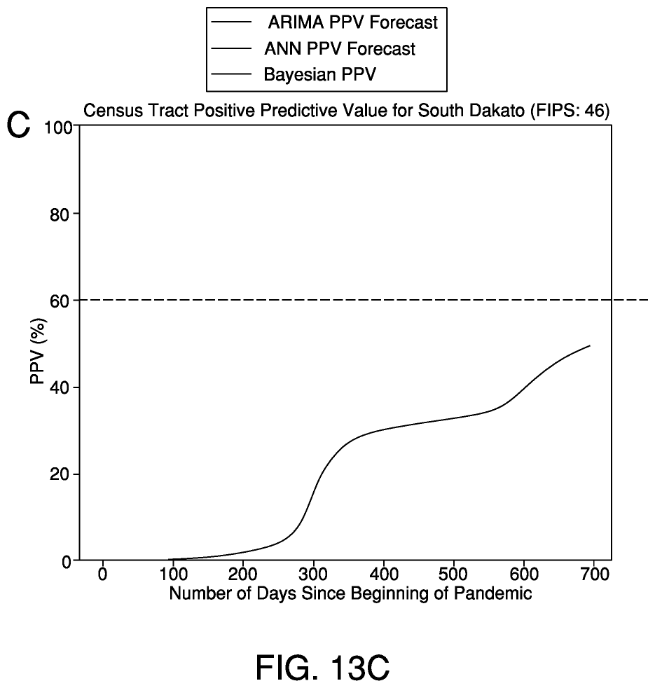

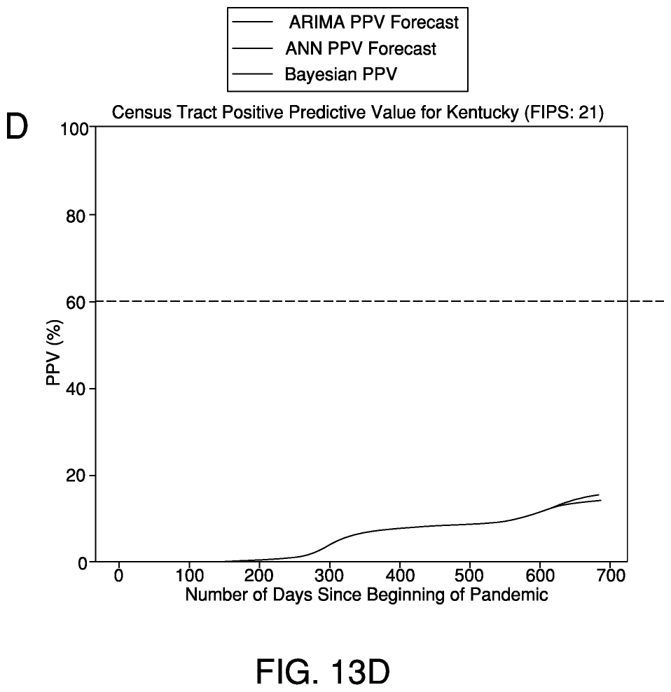

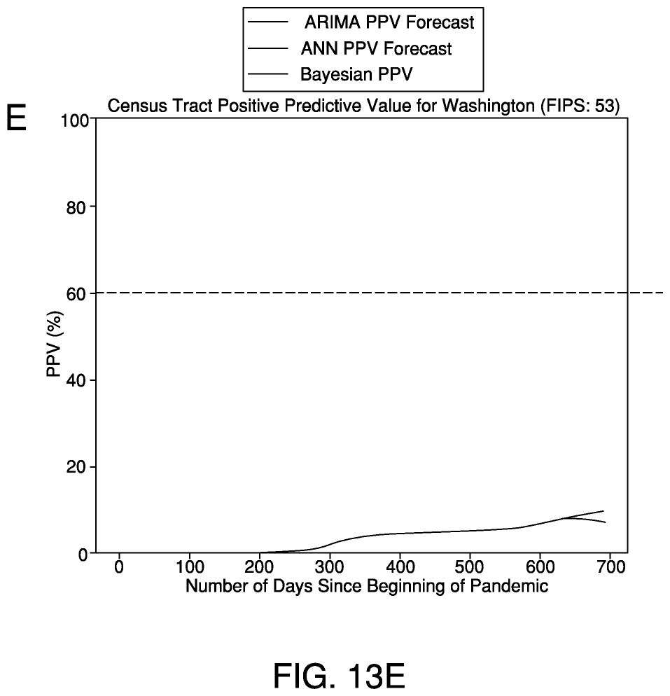

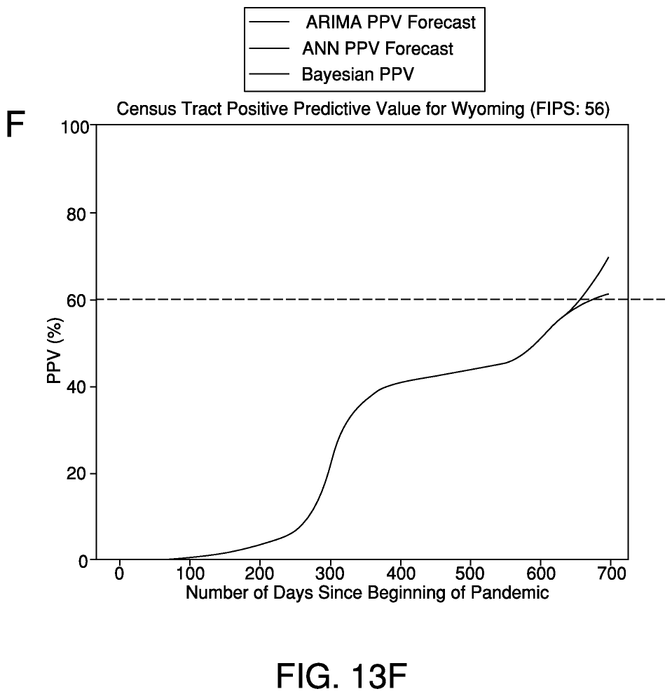

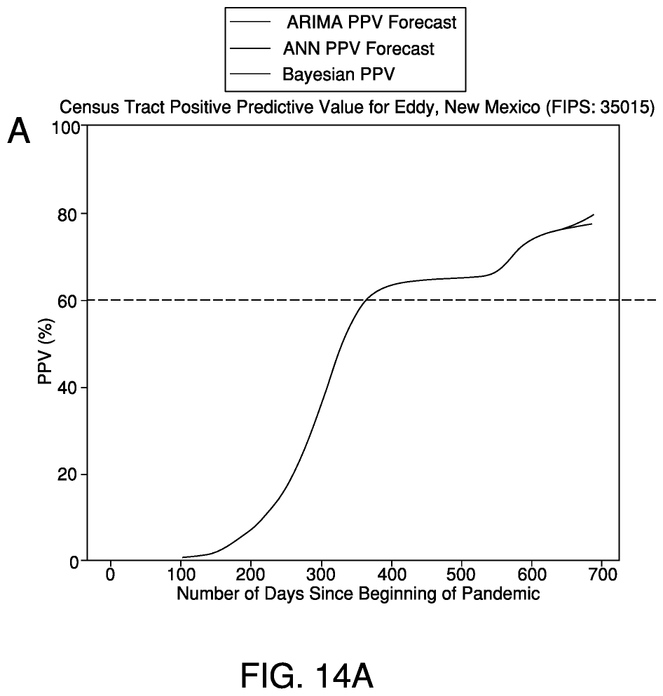

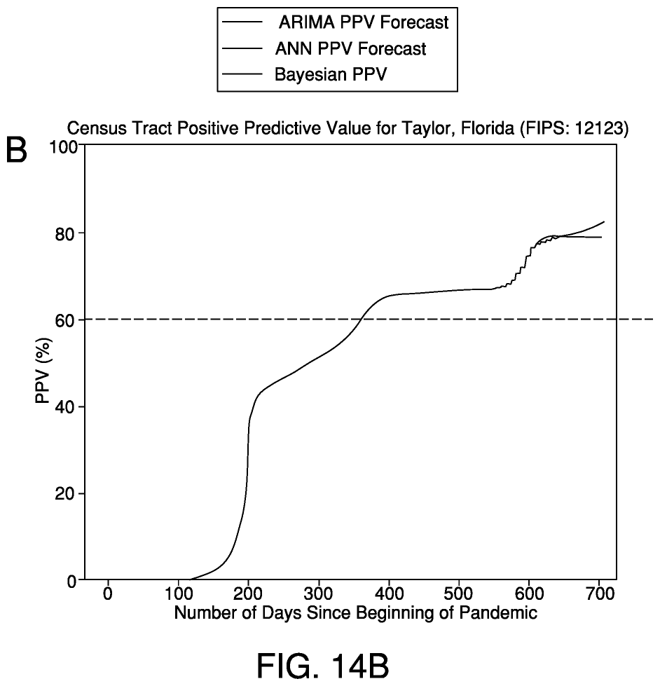

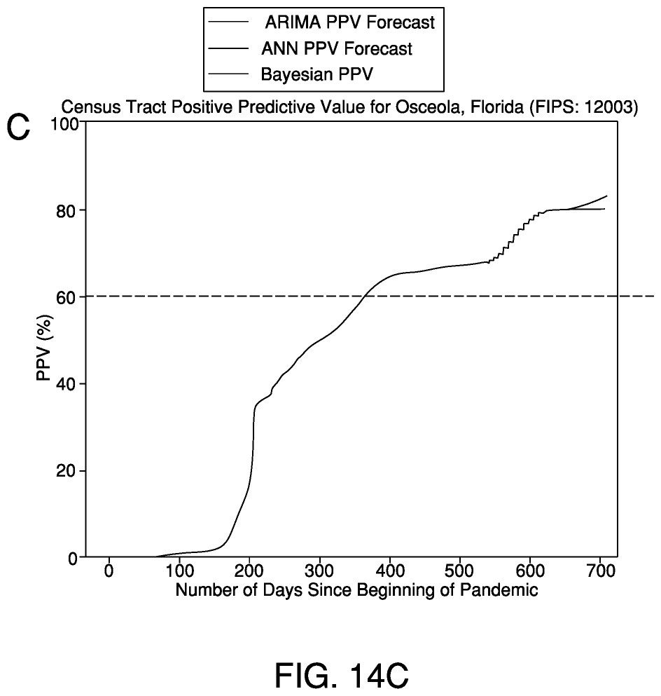

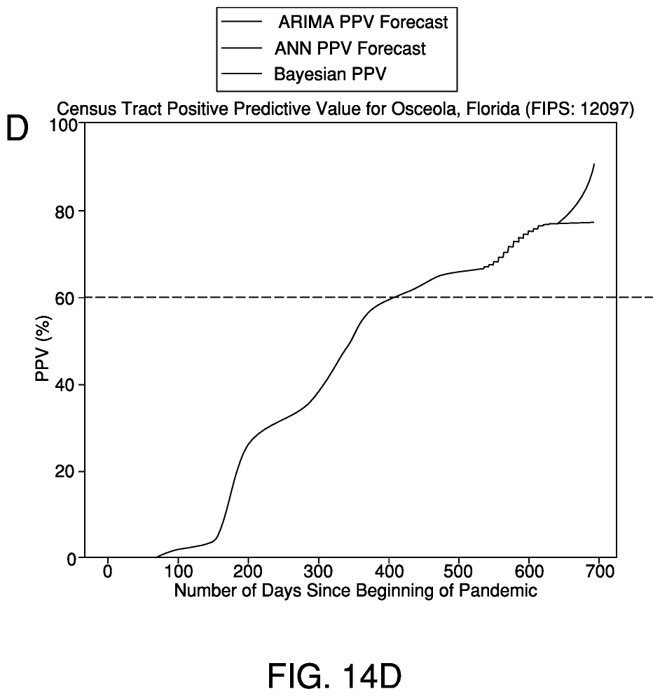

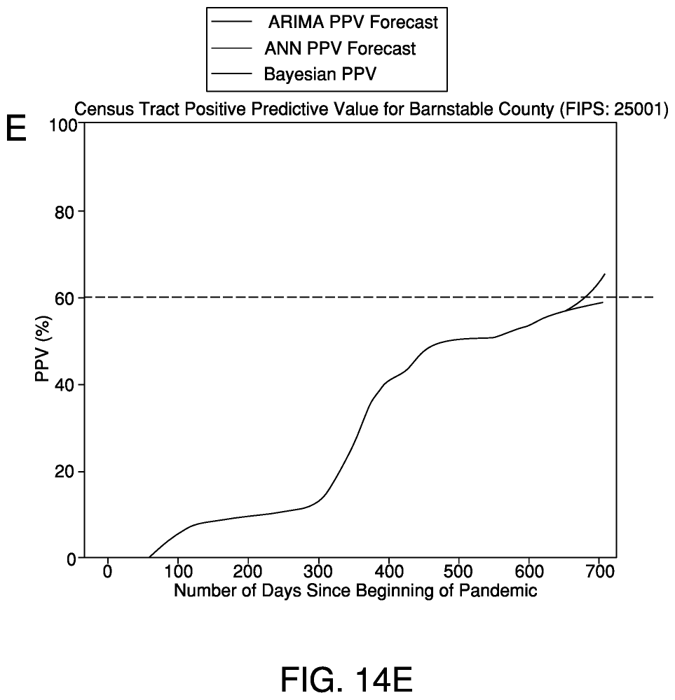

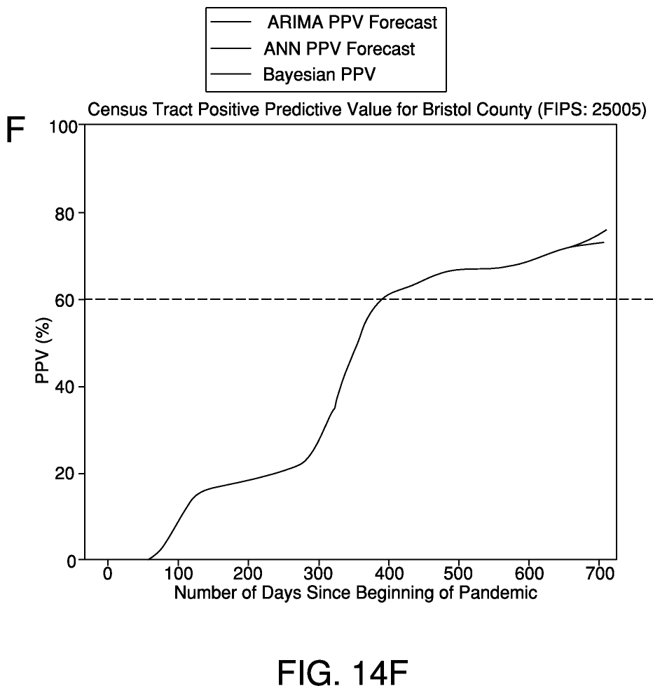

PPV curves were calculated for different counties and states within the United States and found that different regions had very different PPV values over time and at the most recent day in the pandemic. The results for sample states are illustrated in FIGS. 13a-13f, while sample counties are illustrated in FIGS. 14a-14f.

### INTERPRETATION

At the state level (illustrated in FIGS. 13a-13f), the PPV values at the state level were below the 60% cutoff over the course of the pandemic, but for Wyoming it was forecast to cross the 60% threshold in the next 50 days using the ANN forecasting method. This would indicate that the state of Wyoming or a hospital in Wyoming would not need to budget for an additional confirmatory test to be performed after this forecasted date.

At the county level (illustrated in FIGS. 14a-14f) all of the county-level examples showed that the PPV crossed the 60% threshold (in contrast to many of the state examples), except for Barnstable County, which was forecast to cross the threshold in the future 50 days. As with Wyoming, the county or hospitals should repeat their COVID-19 PCR tests. However, within the next 50 days it is predicted that the PPV will rise above 60%, so at that point the tests will not need to be repeated, and testing budgets can be reduced accordingly.

### CONCLUSIONS

This example illustrates that simply calculating the PPV for an entire country like the United States does not account for local epidemiology, and PPV (along with other properties, such as NPV) can vary based on region and the local effects of the pandemic, which can be determined by rates of risk factors in the local community, environmental factors, access to healthcare, etc. This type of model can be used in decision support tools in hospitals and healthcare organizations to determine if additional testing should be done, forecast budget requirements about additional testing, and help communities make individual and public health decisions.

While the invention has been described with reference to a particular embodiment and application, numerous variations and modifications could be made thereto by those skilled in the art without departing from the spirit and scope of the invention as claimed. Accordingly, the scope of the invention should be determined with reference to the claims.

1. A computer-based method for assessing measurement results on a localized basis comprising:
(a) receiving input data to an input module, the input data comprising individual-level data and population-level data, wherein the individual-level data and population-level data are validated, wherein validation of the individual-level data and population-level data comprises checking for missing values; ensuring that data values fall within a permitted range for a given measure; ensuring data are the correct data type; and combinations thereof;
(b) normalizing the individual-level data and the population-level data by a normalization module;
(c) transforming the individual-level data and the population-level data by a transformation module to a format for use by a machine learning module, wherein transforming the individual-level data and the population-level data comprises rescaling the data from 0 to 100, ensuring all values are positive by multiplying negative values by −1, recentering the data, and combinations thereof;
(d) calculating first error parameters (first EPs) and first error statistical parameters (first ESPs) from individual-level data by a programmable processing unit, wherein first EPs and first ESPs comprise a combination of (1) true positives, true negatives, false positives and false negatives from a set of individual-level data; (2) forecasted first EPs and first ESPs from previously calculated first EPs and first ESPs over time for a given set of individual-level data using time series analysis; and (3) prediction from properties of a population over time;
(e) calculating second EPs and second ESPs from population-level data by the programmable processing unit, wherein second EPs and second ESPs comprise a combination of (1) forecasted second EPs and second ESPs from previously calculated second EPs and second ESPs using time series analysis and calculating second EPs and second ESPs for separate demographic subgroups; and (2) prediction from properties of a population;
(f) determining by the machine learning module the likelihood that a measurement result is more likely to be true or false, wherein the machine learning module is programmed using machine learning algorithms or artificial intelligence algorithms to determine whether the measurement result is more likely to be true or false based on a plurality of data points from previous measurement results that previously were deemed to be true, false or not valid;
(g) determining by the programmable processing unit if the likelihood that the measurement result being true or false should be accepted based upon pre-programmed parameters, the pre-programmed parameters comprising the first EPs, the second EPs, the first ESPs, the second ESPs and combinations thereof;
(h) reporting over a communications link the likelihood that the measurement result is more likely to be true or false.

2. The method of claim 1, wherein the measurement comprises a laboratory test result or a diagnostic procedure.

3. The method of claim 2, wherein the first EPs and the second EPs comprise sensitivity, specificity, prevalence, receiver-operator area under the curve (ROAUC), positive predictive value (PPV), negative predictive value (NPV), false discovery rate (FDR), false omission rate (FOR) and combinations thereof.

4. The method of claim 2, wherein the first ESPs and the second ESPs comprise p-values, adjusted p-values, confidence intervals, sample sizes and combinations thereof.

5. The method of claim 2, further comprising executing the machine learning module using individual-level data and population-level data as inputs to learn whether the measurement result is likely to be true or false.

6. The method of claim 5, wherein individual-level data comprise medical tests results; machine-learning predictions; medical triage algorithms; properties, attributes, metadata, or other information assigned to an individual person, object, or other unit of study; location; age; sex; ethnicity; socioeconomic factor; medical records data; habits; activities; occupation; and combinations thereof.

7. The method of claim 6, wherein the medical test results comprise COVID-19 antibody and polymerase chain reaction test results.

8. The method of claim 2, wherein population-level data comprise individual-level data summarized to the population-level; properties, attributes, metadata, or other information assigned to a population of people, groups of object, average laboratory result rate; average machine learning result;
disease rates; unemployment rates; diagnosis rates; crime rates;
environmental features; average distance to physical locations; and
combinations thereof.

9. The method of claim 8, wherein the population-level data is measured for one or more geographical levels, wherein the one or more geographical levels comprise national, subnational, state, county, census tract levels.

10. The method of claim 1, wherein the determination that the measurement result is more likely to be true or false is encoded by an encoding module into a format usable by electronic medical records systems, decision support systems, electronic formats comprising JSON, XML, or CSV, wherein the likelihood that the measurement result is more likely to be true or false is transmitted over an Application Programming Interface (API).

11. The method of claim 1, wherein the method is performed by one or more general purpose computers, customized computing hardware, local computer server, virtual machine, mobile device, tablet, cloud-based device or combinations thereof comprising a programmable processing unit, volatile memory comprising Random Access Memory, non-volatile memory comprising one or more hard disk drives or solid-state disk drives, and one or more network connections to other devices.

12. A computer-based method of determining the test quality of the results of a specific medical test conducted within a population comprising:
performing the method of claim 1 and calculating PPV using Bayesian calculation based on first EPs and first ESPs,
wherein a PPV of greater than a predetermined value indicates that the specific medical test results does not need to be repeated,
wherein a PPV of less than the predetermined value indicates that the specific medical tests performed in the defined region should be repeated.

13. A computer-based method of forecasting the PPV of the test results of a specific medical test within a defined region comprising:
performing the method of claim 1 and calculating PPV for each day within a time period using Bayesian calculation based on first EPs and first ESPs; and
thereafter forecasting PPV for future dates to predict how long the specific medical tests should be repeated,
wherein forecasting PPV comprises statistical regression methods,
wherein a budget for performing the specific medical test within the defined region can be forecast based on the amount of time the specific medical tests should be repeated based on forecasted PPV.

14. The method of claim 13, wherein the statistical regression methods comprise autoregressive integrated moving average (ARIMA) or a neural network (ANN).
